(19) United States
(12) Patent Application Publication
(10) Pub. No.: US 2007/0083260 A1
(43) Pub. Date: Apr. 12, 2007

(54) Intraocular lens spacer for cataract surgery

(76) Inventor: Michael Colvard, Pacific Palisades, CA (US)

Correspondence Address: KOPPEL, PATRICK & HEYBL, 555 ST. CHARLES DRIVE, SUITE 107, THOUSAND OAKS, CA, 91360, US

(21) Appl. No.: 11/245,904

(22) Filed: Oct. 7, 2005

(51) Int. Cl. A61F 2/16

(52) U.S. Cl. 623/6.16; 623/6.15; 623/6.39; 623/6.41

(57) ABSTRACT

A device for maintaining the normal depth of a posterior chamber of the eye. One or more spacers are implantation in the lens capsule of a patient at a position anterior to an implanted posterior intraocular lens following removal of the natural crystalline lens. The spacer or at least one spacer has an outer diameter approximating the diameter of the lens capsule prior to removal of the crystalline lens. The one or more spacers in combination with the intraocular lens produce a depth for the lens capsule approximating the depth of the lens capsule prior to removal of said crystalline lens. The one or more spacers may be configured to prevent epithelial cell migration.

The invention is directed to the use of one or more spacers to be placed anterior to an intraocular lens (IOL), both the spacer and IOL being within the lens capsule, of an aphakic individual. The spacers are useful during any cataract surgery but maybe particularly beneficial when the patient is a high myope.

### BACKGROUND

The human eye comprises a spherical structure that includes a cornea, which comprises the outer surface of the eye, a crystalline lens centrally located in a lens capsule behind a pupil and retina, optic and other nerves on the rear wall of the eye. These nerves connect the eyes to the brain, and particular areas of the brain that are in neural communication with the eyes. Images pass through the cornea and a pupil, which is centrally located in the iris, and are focused by the lens onto the image receptors at the rear of the eye.

Each eye forms an image upon a vast array of light sensitive photoreceptors of the retina. The outer cover of the eye, or cornea, protects the lens and acts as a colorless filter to refract light onto the iris and pupil. The iris corresponds to the aperture in a camera and contains muscles that alter the size of the pupil to control the amount of light that enters the eye. The natural crystalline lens located posterior to the pupil has a variable shape under the indirect control of peripheral ciliary muscles. Having a refractive index higher than the surrounding media, the crystalline lens gives the eye a variable focal length, allowing accommodation to objects at varying distances from the eye. Much of the remainder of the eye is filled with fluids under pressure that help the eye maintain its shape.

The human eye is susceptible to numerous disorders, diseases and optical deficiencies. Corrective glasses, contact lenses or laser sculpting typically addresses optical deficiencies. Besides optical deficiencies, several diseases that can affect the natural crystalline lens or the optical nerve or macula can degrade vision. For example, cataracts interfere with vision by causing a cloudy or opaque discoloration of the natural lens of the eye. Cataracts often result in partial or complete blindness. If this is the case, the crystalline lens can be removed and replaced with an intraocular lens (IOL). As addressed below, cataract lens removal presents addition optical problems and may result in retinal detachment and macular degeneration. This is specifically true for a myopic patient, and in particular one who is highly myopic.

Intraocular lenses (IOLs) have proven to be very successful in restoring normal vision to individuals following removal of a natural crystalline lens clouded by the presence of a cataract. The normal human natural lens is thicker in its center than an IOL and this may present a problem with any patient. In particular, in an individual considered to be a high myope (requiring an optical correction greater than about 5 diopters) the natural lens may have a thickness as great as 5mm. Following implantation of an IOL the posterior lens capsule wall tends to shrink and wrap around the IOL. Because of the greater thickness of the removed lens, the shrinking capsule can result in the loss of several millimeters of capsule depth, and the remaining posterior capsule wall and vitreous fluid shifts forward. This forward movement of the vitreous fluid can cause a retinal detachment and initiate macular degeneration.

This situation was recognized by Giovinazzo in U.S. Pat. No. 4,710,195 wherein he states:
“patients with high myopia are recommended by many to have an implant lens not for optical correction but to prevent the mobility of the posterior capsule. This mobility and subsequent anterior-posterior movement of the vitreous removes many of the benefits of extra capsular surgery.”

However, IOLs have not been found to adequately prevent the forward movement of the rear wall of the capsule.

This problem may be of an even greater concern should the posterior membrane tear or have to be later removed or opened due to posterior capsular opacification (PCO), which is normal in about 30% of IOL placements. PCO can occur due to the proliferation of epithelial cells at the periphery of the posterior capsule wall that can grow and spread under the IOL on the inside surface of the wall and cause opacification. The normal IOL structure may not be adequate to prevent this PCO.

Lenses have been designed with a ring as part of the rear surface of the IOL in an attempt to prevent opacification and forward movement of the vitreous. U.S. Pat. No. 4,244,060 to Hoffer shows a plano-convex posterior chamber lens with a rearward projecting, substantially annular ridge or lip which presses against the rear capsule. The lip is stated to limit “the progress of vitreous humor toward the anterior chamber after a decision, and may limit lens fiber growth on the posterior capsule within the lip region.” Other lens designs are intended to prevent the growth of cells onto the IOL, and thus inhibit posterior capsule opacification, by providing a peripheral wall having an outer corner edge with a sharp outer corner resting against the capsule wall to substantially retard or prevent the growth of cells onto the lens side walls and eventually extending across the rear surface of the IOL.

While not specifically designed for high myopes, another approach is to provide an optic which totally fills the posterior capsules. One approach is shown by Siepser, U.S. Pat. No. 4,556,998 and 5,147,394, which show an expandable hydrogel. A lens of about 2 to 5 mm in diameter and an appropriate thickness is formed from a dry hydrogel. That lens is then implanted in the posterior capsule where the natural fluids wet the hydrogel which swells to a diameter of 6-14 mm along with an increase in thickness which may fill the depth of the capsule.

A still further alternative is to provide an inflatable lens such as shown in U.S. Pat. No. 4,619,662 to Juergens or U.S. Pat. No. 4,822,360 to Deacon. In these designs, an inflatable, transparent sac or bag is placed in the posterior capsule. The bag is inflated to its intended dimension by filling with a fluid, which may be a polymerizable elastomer, to create an optically correct, transparent lens with proper vision correction. These lenses can be made to fill the posterior capsule.

Attempts have also been made to provide special lens designs to meet the optical requirements of high myopes. These include the use of thicker and greater diameter optics, or lenses with a much greater rearward angulation. U.S. Pat. No. 3,866,249 to Flom shows a thick biconvex IOL which is said to provide support for the hyloid membrane and the vitreous humor.

A further alternative which may be used to provide large optical corrections is to implant two lenses in a single eye. The lenses may be separated from each other by a spacer, or a ring shaped frame may be provided with a central circular opening to receive a lens of desired optical characteristics. This lens insert could also be very thick to provide telescopic properties. U.S. Pat. No. 5,769,890 to McDonald is directed to placement of a second IOL, preferably behind the iris but in front of the capsule containing a first IOL, to correct optical errors resulting from the selection of a first, prior implanted IOL. U.S. Pat. No. 6,616,692 and U.S. Pat. No. 6,797,004 to Brady and Glick also show implantation of two IOLs, both providing optical correction. The '004 patent shows a peripheral holder with the lens centrally located therein or two optical lenses separated by an intermediate solid spacer to maintain a preset space between the lenses.

Other examples of peripheral rings to hold an IOL are shown in U.S. Pat. No. 5,628,798 and Published application 2002/0128710 Eggleston, et al. U.S. Pat. No. 6,007,579 to Lipshitz et al shows a telescopic optic held in a circular ring. U.S. Pat. No. 5,876,442 is a further example of a telescopic optic between two spaced apart carrier rings. Other examples of the use of a peripheral ring to hold an IOL are U.S. Pat. No. 5,824,074 to Koch and U.S. Pat. No. 5,628,795 and RE 34,998 to Langerman. These rings are positioned radially outward from the optic and are used to hold the optic and maintain the diameter of the capsule or provide accommodation and are not intended to, and do not function, to space the optic rearwardly to maintain the position of the posterior capsule wall and the volume of the vitreous chamber.

Also, separate rings have been suggested to maintain the normal capsule diameter. U.S. Pat. No. 5,843,184 shows an example of a tension ring placed in the capsule solely for the purpose of maintaining the diameter of the capsule and does not hold or space the optic and will not maintain the capsule volume and vitreous space. Dick discloses the use of a closed, folded, rigid capsular ring inserted prior to IOL Placement to maintain a fixed capsule diameter (Dick, H.B., “Closed Foldable Capsular Rings”, J. Cataract Refract. Surg., 31, pp 467-471 (March 2005).

However, none of these devices have proven to be suitable to maintain capsule depth and vitreous chamber volume and depth unchanged. There is therefore a need for a suitable means for maintaining the shape and volume of the posterior capsule containing an IOL and the position of the rear wall of the capsule in relationship to the rear of the eye, so the vitreous will not move forward, in turn helping the macula and retina to remain intact. This would be useful in all cataract surgery and in particularly in aphakic myopics.

### SUMMARY

Spacers comprising rings or discs for implantation in the posterior lens capsule of an individual anterior to an intraocular lens are described. These spacers aid in maintaining the normal depth of the patient's posterior capsule and to preventing forward movement of the vitreous, and retinal detachment that may occur as a result of such movement. In addition, the spacers may have a particular configuration to impede the spread of epithelial cells to reduce or prevent PCO.

### BRIEF DESCRIPTION OF DRAWINGS

FIG. 1 is a schematic cutaway side view of the human eye.

FIG. 2 is a schematic cutaway side view showing an IOL located in the posterior capsule of a human eye.

FIG. 3 is a schematic cutaway side view of an IOL placed in the posterior capsule along with spacers incorporating features of the invention positioned anterior to the IOL.

FIG. 4 is a schematic cutaway side view of an IOL placed in the posterior capsule along with second arrangement of multiple spacers positioned in the capsule anterior to the IOL.

FIG. 5 is a side perspective view of a disc incorporating features of the invention.

FIG. 6 is a side perspective view of a first embodiment ring incorporating features of the invention.

FIG. 7 is a side view of a modification of FIG. 6 showing a ring with angled lower surface cutaway along line 7-7 of FIG. 6 second embodiment of a ring.

FIG. 8 is a cross sectional view of a combination of an IOL, angled ring and disks as it would appear in a posterior capsule.

FIG. 9 is a schematic cutaway view of a further embodiment showing an 10L plus multiple spacers.

FIG. 10 is a cross sectional view of a further embodiment of an 10L with a ring and spacer.

### DETAILED DESCRIPTION

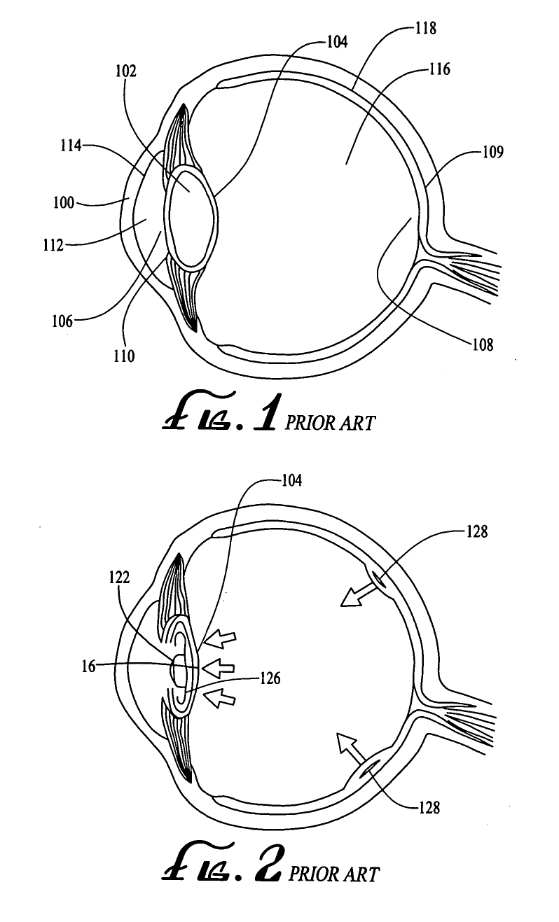

The structure of the human eye is shown schematically in FIG. 1. The human eye comprises a spherical structure which includes the cornea 100, which comprises the outer surface of the eye, a crystalline lens 102 centrally located in a lens capsule 104 behind the pupil 106 and the retina, optic and other nerves 108 on the rear wall of the eye that connect the eyes to the brain, and particular areas of the brain that are in neural communication with the eyes. Images pass through the cornea 100 and iris 110, which is centrally located in the pupil 106, and are focused by the lens 102 onto the image receptors at the rear of the eye.

Each eye forms an image upon a vast array of light sensitive photoreceptors of the retina. The outer cover of the eye, or cornea, protects the lens and acts as a colorless filter to refract light onto the iris and pupil. The iris 110 corresponds to the aperture in a camera and contains muscles that alter the size of the pupil to control the amount of light that enters the eye. The natural crystalline lens 102 located just behind or posterior to the pupil 106 has a variable shape under the indirect control of the peripheral ciliary muscles. Having a refractive index higher than the surrounding media, the crystalline lens gives the eye a variable focal length, allowing accommodation to objects at varying distances from the eye.

Much of the remainder of the eye is filled with fluids under pressure that help the eye maintain its shape. For example, the aqueous humor (fluid) 112 fills the anterior chamber 114 between the cornea 100 and the pupil 106, and the vitreous humor (gel) 116 fills the majority of the volume of the eye in the vitreous chamber 118 that is located between the lens 102 and the retina and other optic nerves 108.

The human eye is susceptible to numerous disorders, diseases and optical deficiencies. Corrective glasses, contact lenses or laser sculpting typically addresses optical deficiencies. Besides optical deficiencies, several diseases that can affect the natural crystalline lens or the optical nerve 108 or macula 109 can degrade vision. For example, cataracts interfere with vision by causing a cloudy or opaque discoloration of the natural lens 102 of the eye. Cataracts often result in partial or complete blindness. If this is the case, as shown in FIG. 2 the crystalline lens can be removed and replaced with an intraocular lens (IOL) 16. Cataract lens removal in a myopic patient, particularly a high myopic, presents addition optical problems and may result in retinal detachment and macular degeneration.

Intraocular lenses (IOLs) 16 have proven to be very successful in restoring normal vision to individuals following removal of a natural crystalline lens 102 clouded by the presence of a cataract. While there are many different designs of IOLs and various different placement procedures, methods for positioning and retaining the IOL in position once placed and various different locations for placement of the lens, a typical state of the art IOL 122 is placed within the lens capsule 104 (which is a membrane bag enclosing the natural lens 102) after removal of the clouded natural lens. While posterior chamber IOLs 122 may have different shapes and dimensions for the IOL optical portion and peripheral retaining structure, a typical posterior IOL 122 has an optic diameter of about 6mn and an optic thickness of about 1.5mm thick.

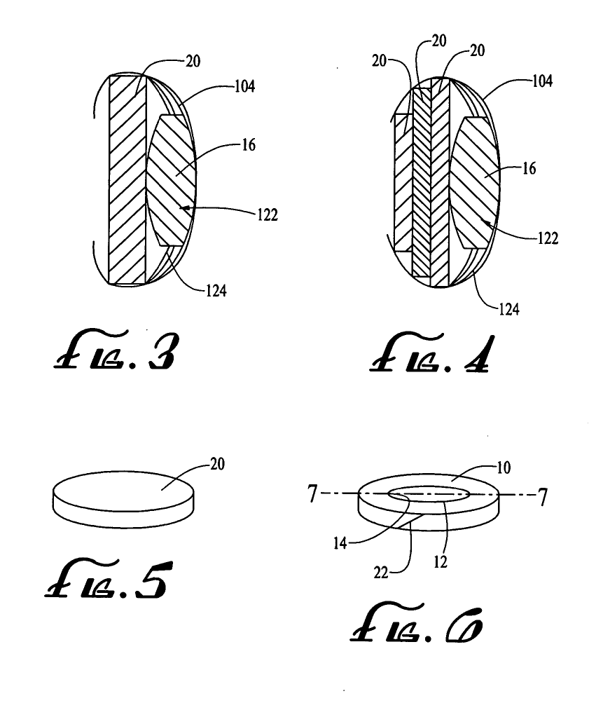

Because a typical lens capsule 104 has a mean diameter of about 10.5mm (range 10.65-12.0mm), the IOL also typically has haptics, flanges or other positioning structure 124 extending outward from the optical portion 16 of the IOL to keep the optical portion 16 centered within the lens capsule 104 with a central axis generally coinciding with an axis through the center of the iris. As shown in FIG. 3, the haptics, flanges or other positioning structure 124 may also be mounted at an angle to the plane of the optical portion 16 of the IOL 122 so that the optical portion 16 is pushed rearward against the remaining membrane (referred to as the posterior or hyloid membrane) 126 forming the rear of the lens capsule 104.

While the normal human natural lens 102 is thicker in its center than an IOL optic 16, this may not present a problem. However, in some individuals and in particular individuals considered to be a high myopic (requiring an optical correction greater than about 10 diopters) the natural lens 102 may have a thickness as great as 5mm. Following implantation of an IOL 122 the posterior capsule 104 tends to shrink and wrap around the IOL 122. Because of the greater thickness of the removed lens 102, the shrinking capsule can result in the loss of several millimeters of capsule 104 depth, the remaining posterior capsule 104 shifts forward, aqueous fluid 112 flows from the anterior chamber 114 through the iris 110 filling the remaining volume of the capsule 104 and vitreous gel 116 posterior of the capsule membrane 126 may move forward.

The arrows within the eye structure shown in FIG. 2 represent the forward movement of the capsule and the vitreous. This forward movement of the vitreous 116 can cause a retinal detachment 128 and initiate macular degeneration. However, IOLs 122 have not been found to adequately prevent the forward movement of the rear wall of the capsule.

This problem may be of an even greater concern should the posterior membrane 126 tear or have to be later opened or removed due to posterior capsular opacification, which is normal in about 30% of IOL placements. The lens structure may not be adequate to prevent vitreous 116 from flowing around the IOL 122 and entering the posterior capsule 104. PCO can occur due to the proliferation of epithelial cells at the periphery of the posterior capsule wall that can grow and migrate under the IOL on the inside surface of the wall and cause opacification. The normal IOL structure may not be adequate to prevent this PCO.

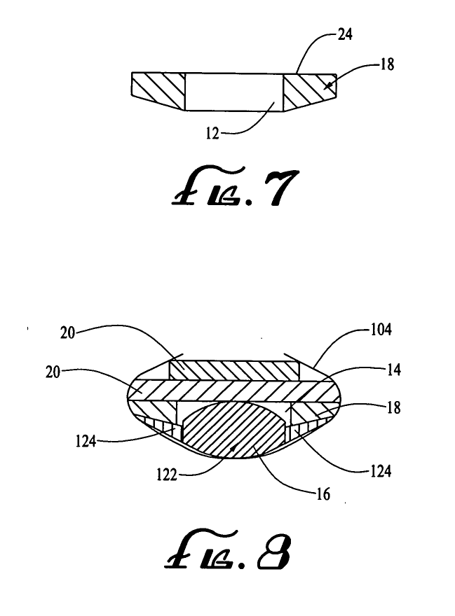

The invention is directed to devices that can be used to position an IOL more rearward in the posterior capsule 104 to maintain capsule dimensions such as shown in FIGS. 3, 4 and 8. The devices comprise one or more spacers which may be in the form of a ring 10, 18 with a central hole 12 having a diameter 14 approximating that of the IOL optic 16 as shown in FIGS. 6 and 7. The device may also be a disk 20, such as shown in FIG. 5, that covers both the optic 16 and the haptic 124 of the IOL.

The collapse of the enlarged capsule of a high myope following cataract removal and IOL placement in the capsule is addressed by placement of one or more transparent rings 10, 18, plates or discs 20 against the anterior face of a posterior chamber IOL 122 and/or the haptic 124 thereof. If rings 10, 18 are used, the centrally located open space 12 within the ring 10 typically has a diameter 14 approximating the diameter of the IOL optic 16, generally greater then about 6mm, the ring 10, 18 being located anterior of the IOL optic 16 and the haptic 124. Alternatively, one or more discs 20 can be stacked on the anterior surface of the IOL but still within the capsule 104.

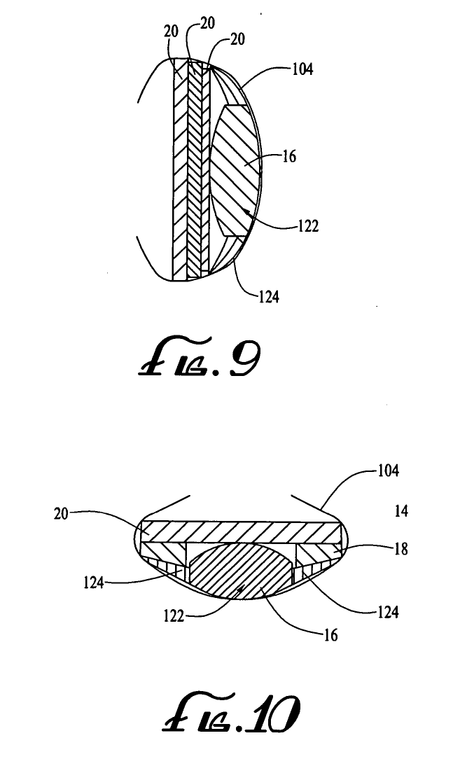

Still further, a combination of discs 20 and rings 10, 18 can be used such as shown in FIGS. 8 and 10. These discs 20 or rings 10, 18 serve as spacers and are not intended to provide any optical correction. Their primary purpose is to maintain the normal depth of the capsule 104, push the IOL optic 16 rearward so that it makes uniform contact with the posterior membrane 126 of the capsule and maintains the normal location of the capsule membrane 126 so that the vitreous 116 does not move forward, which can cause retinal detachment and start macular degeneration. The ring 10, 18 or disk 20 can also have square edges to act as a barrier to prevent migration of epithelial cells along the back of the IOL 122 and thus prevent or retard posterior capsular opacification.

Since the discs and/or rings push the IOL rearward away from the typical central position for an IOL, the optical correction provided by the IOL must be adjusted to compensate for the changed position and provide for the proper optical correction for the patient.

FIG. 5 is a schematic drawing showing an embodiment of a disc 20 incorporating features of the invention. FIG. 6 is a schematic drawing showing a first embodiment of a ring 10 having parallel faces. The spacers, whether in the form of a disc 20, or ring 10, 18, may be constructed of numerous biomaterials typically used for ophthalmic implants including, but not limited to, rigid biocompatible materials such as polymethyl methacrylate (PMMA) or polycarbonate or, preferably, deformable materials such as silicone, acrylic or hydrogel polymeric materials, and the like. If a single spacer (disc 20 or ring 10, 18) is used, a typical spacer would have an outer diameter to match the normal diameter of the patients capsular bag 104 when enclosing a natural lens 102, typically 9.5-13mm, and a thickness of from about 0.5 to about 3 mm. If multiple rings 10, 18 or discs 20 are used the thickness of the largest diameter spacer, which is preferably the spacer contacting the anterior surface of the IOL 122, can be reduced to compensate for the thickness of the other spacers. The other spacers would typically have smaller diameters so that they form a truncated pyramid when stacked on top of each other without spaces there between, such as shown in FIGS. 4 and 8. While shown in the figures as a planer disc, when implanted the planer spacer contacting the IOL would preferably contour to the surface of the IOL. This would occur without effecting the optical correction provided by the IOL. A typical single ring 10, 18 or disc 20 has an outer diameter of about 11-12 mm and a thickness from about 0.5mm to about 3 mm, but may be thicker if the posterior chamber is unusually deep. If multiple discs 20 or rings 10, 18 are used they can have different thicknesses but the total thickness of a stack of discs or rings would chosen to reproduce, in combination with the IOL thickness, the normal depth of the capsular chamber in the patient. Alternatively, as shown in FIG. 9, the spacers can be stacked with the smallest diameter disc against the 10L or the largest disc may be between smaller diameter discs. In other words, the various diameter spacers can be stacked so the outer contour of the stack matches the natural internal contour of the capsule.

When rings are used, it is preferred that the central hole 12 there through is equal to or greater than the diameter of the IOL optic 16 so that the inner edge of the ring 10, 18 is not within the optical path of an image being observed through the IOL 122 as this can distort the image and cause glare. Also, because the ring 10 does not directly push on the optic 16 but instead moves the optic 16 rearward by pressing on the haptic 124, the ring 18, as best shown in FIG. 7, can also have an angled rear surface, for example matching the angle of the haptic 124 from the plane of the optic 16. FIG. 7 shows a ring 18 with an angle to match that of an angled haptic 124, typically about 6°.

On the other hand, discs 20 preferably have parallel front and rear surfaces so that they do not add to or subtract from the optical characteristics of the IOL 122. While they press against the IOL optic 16, which may cause the optic 16 to be spaced rearward, the primary intended function is to also space the haptic 124 rearward, causing rearward movement of the optic 16. For this reason, there is value in combining a ring 10, 18 with a disc 20 and particularly the ring 18 with angled rear surface such as shown in FIGS. 7, 8 and 10.

While multiple discs are shown, the same function can be obtained by use of a single disc and/or ring combination that duplicates the configuration of the multiple discs. For example, while FIGS. 4 and 9 show three stacked discs, a single disc can provide this configuration with a stepped edge or a contoured edge that approximates the contour of the capsule in which it is to be placed. In the same manner, the ring and two discs shown in FIGS. 8 or 10 can be provided as a single spacer having the same or similar outer contour and a posterior opening to receive the optic of the IOL.

As a further variation, the disc 20 or ring 10, 18 does not have to be a solid material. It can be an inflatable disc 20 or ring 10, 18 that can be filled with a liquid before or after placement to create the desired spacer dimensions. This can also provide an opportunity to vary the dimensions of the spacer once implanted by adding or removing the filler material.

As a still further variation, if the disc 20 or ring 10, 18 is flexible, it may also provide accommodation if the zonules in the eye are still intact, causing the IOL optic 16 to move in response to the eye trying to focus on images at different distances. While the rings and disc are shown as solid structures, the same spacing effect can be obtained by proving a notch in the disc or a slot across the ring providing an opportunity for the disk to contour more readily to the IOL or the ring diameter to be increased or decreased after implantation. In such instance, to prevent cell migration through the slot of notch it is preferred to use two or more of the spacers with the notch or slots oriented in different directions to present a tortuous movement path for the migrating cells. As a still further alternative, the ring can be provided with an angled cut 22 through the toroidal portion 24 of the ring so that the surfaces along the angled cut can be displaced as the diameter of the ring is increased or decreased. This allows for slight variation in the ring diameter without providing access for cell migration.

While the invention may have specific benefit for use in myopic patients who may have a lens capsule with a greater depth then normal, it is also contemplated that the devices shown and described herein can be used in patients with normal capsule dimensions because IOL lenses are usually of a lesser depth (thickness) than the natural crystalline lens which the IOL replaces. One skilled in the art will recognize that, based on the disclosure herein, variations on the construction and shape of the spacers can be made without varying from the invention disclosed herein and the invention is limited only by the claims set forth below.

1. A device for maintaining the normal depth of a posterior chamber of the eye comprising one or more spacers for implantation in a lens capsule of said individual anterior to a posterior intraocular lens implanted following removal of a natural crystalline lens, said
posterior intraocular lens comprising an optic portion and optionally a haptic portion extending from the optic portion, the spacer or at least one spacer having an outer diameter approximating the diameter of the lens capsule prior to removal of said crystalline lens, the one or more spacers in combination with the intraocular lens producing a depth for the lens capsule approximating the depth of the lens capsule prior to removal of said crystalline lens.

2. The device of claim 1 wherein the one or more spacers are configured to prevent epithelial cell migration.

3. The device of claim 1 wherein the one or more spacers comprises one or more discs, one or more rings, a combination of one or more discs and one or more rings or a single spacer configured to simulate multiple discs, rings or a combination of multiple discs or rings in a single structure.

4. The device of claim 3 wherein the at least one ring has an opening extending axially at least partially there through, said opening having a diameter at least as great as the diameter of the optic portion of said intraocular lens.

5. The device of claim 3 wherein the at least one ring has an angled posterior surface, said angled posterior surface being angled to substantially match the angle of the haptic extending from the optic portion of the intraocular lens.

6. The device of claim 3 comprising two or more discs, each disc having a different diameter, the discs stacked so that the diameter of each disc approximates the diameter of the lens capsule at a location where placed in said capsule, an appropriate diameter disc placed against the anterior surface of the optic or haptic portion of the intraocular lens.

7. A device for prevention of forward movement of the vitreous and retinal detachment following removal of a crystalline lens and placement of a posterior chamber intraocular lens in a posterior chamber of the eye comprising one or more spacers sized for implantation in the lens capsule of said individual anterior to said posterior intraocular lens, said one or more spacers having a diameter approximating the diameter of the lens capsule prior to removal of said crystalline lens, the thickness of the one or more spacers in combination with the thickness of the intraocular lens producing a depth for the lens capsule approximating the depth of the lens capsule prior to removal of said crystalline lens.

8. The device of claim 7 wherein the one or more spacers are configured to prevent epithelial cell migration.

9. The device of claim 7 wherein the one or more spacers comprises one or more discs, one or more rings, a combination of one or more discs and one or more rings or a single spacer configured to simulate multiple discs, rings or a combination of multiple discs or rings in a single structure.

10. The device of claim 9 wherein the at least one ring has an opening extending axially at least partially there through, said opening having a diameter at least as great as the diameter of the optic portion of said intraocular lens.

11. The device of claim 9 wherein the at least one ring has an angled posterior surface, said angled posterior surface being angled to substantially match the angle of the haptic extending from the optic portion of the intraocular lens.

12. The device of claim 9 comprising two or more discs, each disc having a different diameter, the discs stacked so that the diameters thereof progress in accordance with the diameters of the lens capsule, the appropriate diameter disc placed against the anterior surface of the optic or haptic portion of the intraocular lens.

13. The device of claim 1 or 7 wherein the one or more spacers comprise rigid or deformable biocompatible materials.

14. The device of claim 13 wherein the biocompatible material is a rigid polymethyl methacrylate or polycarbonate material or a deformable silicone, acrylic or hydrogel polymer.

15-20. (canceled)
